(19) United States
(12) Patent Application Publication
(10) Pub. No.: US 2023/0307144 A1
(43) Pub. Date: Sep. 28, 2023

(54) Method, computer equipment, storage media and program products for obtaining the Index of Microvascular Resistance

(71) Applicant: ArteryFlow Technology Co., Ltd., Hangzhou (CN)

(72) Inventors: Jingsong HE, Hangzhou (CN); Xiaochang LENG, Hangzhou (CN); Jianping XIANG, Hangzhou (CN)

(21) Appl. No.: 18/125,589

(22) Filed: Mar. 23, 2023

(30) Foreign Application Priority Data
Mar. 23, 2022 (CN) 202210292825.X

(51) Int. Cl. G16H 50/50; A61B 5/026

(52) U.S. Cl. CPC G16H 50/50; A61B 5/026; G06T 2207/30104

(57) ABSTRACT

This application discloses a method, a computer device, a storage medium, and a program product for obtaining the Index of Microvascular Resistance (IMR). The method comprises the following steps: obtaining the resting pressure of the coronary artery ostium, and calculating the cardiac output pressure based on the resting pressure of the coronary artery ostium. Obtaining the hyperemic pressure of the coronary artery ostium, and obtaining the coronary flow reserve based on the cardiac output pressure, the resting pressure, and the hyperemic pressure. Obtaining the medical images of the coronary system, obtaining the three-dimensional model of the target vessel, and calculating the morphological parameters of the coronary artery based on the three-dimensional model of the target vessel. Calculating the resting blood flow and the flow resistance based on the morphological parameters.

### CROSS-REFERENCE TO RELATED APPLICATIONS

This application claims priority from the Chinese patent application 202210292825.X filed Mar. 23, 2022, the content of which is incorporated herein in the entirety by reference.

### TECHNICAL FIELD

This application relates to the field of medical image data processing technology, particularly to a method, computer equipment, storage media and program products for obtaining the Index of Microvascular Resistance.

### BACKGROUND

The heart is one of the most important organs in the human body, and it serves as the driving force for blood circulation. When the heart contracts, blood flows from the left ventricle through the aorta and its various branches to the capillaries of all tissues in the body, providing blood supply to the entire body. At the same time, the heart itself needs to be supplied with blood by the coronary artery system, which consists of epicardial arteries and microvascular system. Coronary microvascular circulation refers to the microvascular system composed of arterioles, venules, and capillaries in the coronary artery, which is the main site for material exchange between tissue cells and blood. In the current technology, it is not possible to directly observe the coronary microvascular system through imaging, and only specific parameters can reflect microvascular function.

Coronary flow reserve (CFR) is defined as the ratio of the hyperemic blood flow in the coronary artery to the resting blood flow, which reflects the ability of the coronary artery to increase blood flow when the oxygen demand of the body increases. It takes into account the blood supply from the epicardial arteries and microvascular.

Fractional flow reserve (FFR) is defined as the ratio of the maximum blood flow that the myocardium can obtain in the presence of stenosis to the theoretically maximum blood flow that the myocardium can obtain in a normal state. It reflects the blood supply function of the epicardial arteries and is mainly used for functional evaluation of coronary stenosis.

The Index of Microvascular Resistance (IMR) is defined as the product of the coronary distal pressure and the mean transit time in hyperemia, which reflects the blood supply function of the coronary microvascular and is mainly used for the evaluation of coronary microvascular circulation.

In current clinical practice, CFR, FFR, and IMR are important indicators for functional evaluation of the coronary system, which can systematically reflect the blood supply function of the coronary system. However, the current clinical practice generally uses a pressure wire to measure these parameters, which involves difficulties in operation and high cost.

### SUMMARY

The present application provides a method for obtaining the Index of Microvascular Resistance. The method for obtaining the Index of Microvascular Resistance includes the following steps:

Obtaining the resting pressure of the coronary artery ostium, and calculating the cardiac output pressure based on the resting pressure of the coronary artery ostium.

Obtaining the hyperemic pressure of the coronary artery ostium, and obtaining the Coronary flow reserve based on the cardiac output pressure, the resting pressure of the coronary ostium, and the hyperemic pressure of the coronary artery ostium.

Obtaining medical images of the coronary artery system, extracting the vascular boundary of the coronary artery from the medical images, obtaining the target vessel's 3D model, and calculating the morphological parameters of the coronary artery based on the 3D model of the target vessel.

Calculating the resting blood flow based on the morphological parameters.

Calculating the flow resistance based on the morphological parameters and the resting blood flow.

Calculating the average blood flow of the epicardial vessels in hyperemia based on the Coronary flow reserve and resting blood flow, and calculating the Fractional Flow Reserve and the pressure at the distal of the target vessel in hyperemia based on the morphological parameters, average blood flow in hyperemia, and hyperemic pressure of the coronary artery ostium.

Calculating the Index of Microvascular Resistance based on the morphological parameters, average blood flow of the epicardial vessels in hyperemia, and pressure at the distal of the target vessel.

Optionally, the cardiac output pressure is calculated by the following formula:

P=a*Pa_rest, where:

P is the cardiac output pressure.

α is the pressure correction factor.

Pa_rest is the resting pressure of the coronary artery ostium.

Optionally, the Coronary flow reserve is calculated by the following formula:

$$CFR = \frac{P - Pa\_hyp}{P - Pa\_rest} ,$$

where:

CFR is the Coronary flow reserve.

Pa_hyp is the hyperremic pressure of the coronary artery ostium.

Optionally, based on mentioned morphological parameters and mentioned resting blood flow, the flow resistance is calculated, specifically comprising:

Obtaining the viscous resistance and expansion resistance based on mentioned morphological parameters, and calculating the flow resistance by combining with mentioned resting blood flow.

Optionally, the morphology parameters include target vessel length, vessel radius, reference vessel area, and narrowed vessel area. The flow resistance is calculated by the following formula:

r2=rᵥ+rₑ*q2, where:

r2 is the flow resistance.

rᵥ is the viscous resistance, which is related to the vessel morphology.

rₑ is the expansion resistance, which is related to the degree of vessel stenosis.

q2 is the resting blood flow.

The viscous resistance is obtained by the following formula:

$${r}_{v} = \frac{8 μ L}{π {R}^{4}} ,$$

where:

μ is the blood viscosity coefficient.

R is the vessel radius.

L is the target vessel length.

The expansion resistance is obtained by the following formula:

$${r}_{e} = \frac{p {K}_{e}}{2 {A}_{0}^{2}} {( \frac{{A}_{0}}{{A}_{s}} - 1 )}^{2} ,$$

where:

ρ is the blood density.

A₀ and Aₛ are the reference and stenotic cross-sectional areas, respectively.

Kₑ is a expansion resistance correction coefficient related to the stenosis length, with a range of 1.2 to 2.1.

Optionally, the method for obtaining the Index of Microcirculatory Resistance further comprises: Obtaining the pressure drop of the epicardial vessels based on the average blood flow of the epicardial vessels in hyperemia, viscous resistance, and expansion resistance.

The pressure drop of the epicardial vessels is obtained by the following formula:

DP2=(rᵥ+rₑ*Q2)*Q2, where:

DP2 is the pressure drop of the epicardial vessels.

rᵥ is the viscous resistance, which is related to the vessel morphology.

rₑ is the expansion resistance, which is related to the degree of vessel stenosis.

Q2 is the average blood flow in hyperemia.

Optionally, the Fractional Flow Reserve is obtained by the following formula:

FFR=(Pa_hyp−(rᵥ+rₑ*Q2)*Q2)/Pa_hyp, where:

rᵥ is the viscous resistance, which is related to the vessel morphology.

rₑ is the expansion resistance, which is related to the degree of vessel stenosis.

Q2 is the average blood flow in hyperemia.

Pa_hyp is the hyperemic pressure of the coronary artery ostium.

Optionally, the Index of Microvascular Resistance is obtained by the following formula:

IMR=S*L*Pd_hyp/Q2, where:

IMR is the Index of Microvascular Resistance.

S is the average cross-sectional area of the target vessel.

L is the length of the target vessel.

Pd_hyp is the pressure at the distal of the target vessel in hyperemia.

Q2 is the average blood flow in hyperemia.

This application also provides a computer device for obtaining the Index of Microvascular Resistance, comprising a memory, a processor, and computer programs stored in the memory. The processor executes the computer program to implement the steps of the method for obtaining the Index of Microvascular Resistance as described in this application.

This application also provides a computer storage medium on which computer programs are stored. The computer programs, when executed by a processor, implement the steps of the method for obtaining the Index of Microvascular Resistance as described in this application.

This application also provides a computer program product comprising computer instructions. When executed by a processor, the computer instructions implement the steps of the method for obtaining the Index of Microvascular Resistance as described in this application.

The method for obtaining the Index of Microvascular Resistance provided by this application has at least the following effects: the method for obtaining the Fractional Flow Reserve only requires inputting the coronary medical image and the pressure of coronary artery ostium, without the use of temperature/pressure guidewires for measurement. The streamlined process speeds up the measurement, greatly shortens the operation time, reduces the operation cost, and has significant clinical value. While ensuring measurement accuracy, it reduces the trauma suffered by patients, lowers the difficulty of the operation, and saves operation costs.

### BRIEF DESCRIPTION OF THE DRAWINGS

FIG. 1 shows the schematic diagram of the coronary circulation hemodynamic model structure established in one embodiment of this application.

FIG. 2 shows a three-dimensional model of the target vessel obtained in one embodiment of this application.

FIG. 3 shows an example of a starting frame of medical images of the coronary system in one embodiment of this application.

FIG. 4 shows an example of an ending frame of medical images of the coronary system in one embodiment of this application.

FIGS. 5-6 show the schematic diagram of the IMR calculation process in one embodiment of this application.

FIG. 7 shows the flowchart of the method for obtaining the the Index of Microvascular Resistance in one embodiment of this application.

FIG. 8 shows the internal structure diagram of the computer device in one embodiment of this application.

### DETAILED DESCRIPTION OF THE EMBODIMENTS

Currently, pressure wire is widely used in clinical practice to measure IMR, but it has the drawbacks of high difficulty and cost. Especially for the measurement of the Index of Microvascular Resistance, the most widely used method in clinical practice is temperature dilution method. Firstly, this method requires reaching the maximum hyperemia, which is an invasive examination technique. Secondly, multiple injections of saline solution are required during the measurement process, which increases the difficulty of operation and prolongs the examination time, presenting significant challenges for both patients and operators. On the other hand, different coronary arteries (LAD, LCX, RCA) have different geometric lengths, and the difference in the placement of the pressure wire will directly affect the measurement of Tmn, resulting in low reproducibility of IMR measurement. Currently, the standard used in clinical practice is not less than 75 mm, which means that the distance between the placement of the pressure wire and the catheter ostium should be at least 75 mm, but this standard does not take into account the differences between different vessels.

In order to further illustrate the purpose, technical solutions, and advantages of the present application, the following detailed description is provided in conjunction with the accompanying FIGS. and embodiments. It should be understood that the specific embodiments described herein are only intended to explain the present application and are not intended to limit the present application.

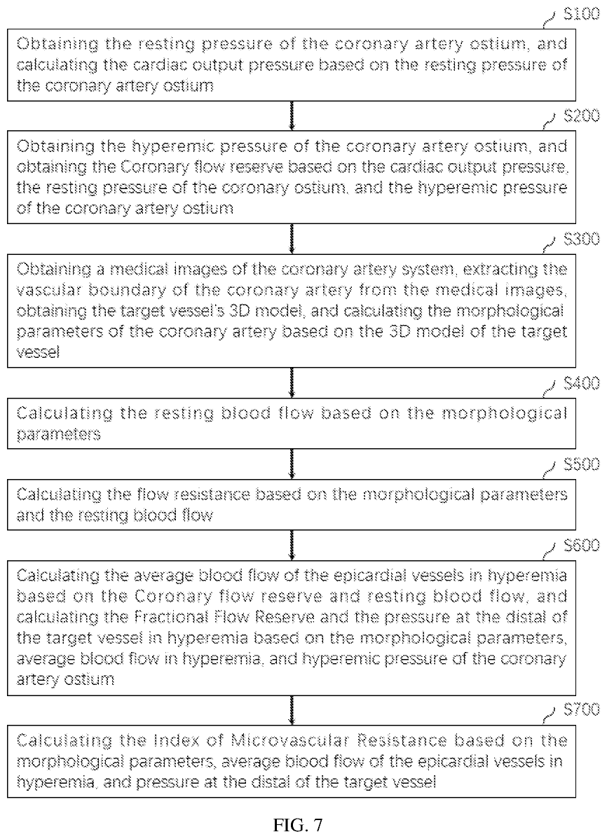

Please refer to FIG. 7. The main steps of various embodiments in this application include:

Step S100: Calculation of cardiac output pressure.

Step S200: Obtaining Coronary flow reserve (CFR).

Step S300: Three-dimensional model reconstruction.

Step S400: Calculation of resting blood flow.

Step S500: Calculation of flow resistance.

Step S600: Obtaining Fractional flow reserve (FFR).

Step S700: Obtaining the Index of Microvascular Resistance (IMR).

The method for obtaining the Index of Microvascular Resistance in this application only requires input of coronary medical images and pressure of coronary artery ostium, without the need of temperature/pressure guide wires for measurement. The simplified process and increased speed greatly shorten surgical time, reduce surgical costs, and have great clinical value.

The methods provided in various embodiments of this application do not require the use of temperature/pressure wires and reduce patient trauma, decrease surgical complexity, and save surgical costs while ensuring the accuracy of the measurement. If the input is static data, the injection of vasodilators such as adenosine into the patient's coronary arteries can also be avoided, further reducing harm to the patient and addressing the issue of some patients being unresponsive to adenosine.

The steps involved in the method provided in this application can be summarized as follows: Firstly, based on the patient's specific coronary artery ostium pressure Pa, and clinical statistics of normal functional parameters, estimate the cardiac output pressure P. Secondly, based on medical imaging techniques such as DSA, CTA, MRA, reconstruct the three-dimensional model of the coronary artery, as well as the resting average blood flow. Thirdly, based on the three-dimensional model, estimate the flow resistance of the the target vessel. Next, based on the pressure change from the patient's resting pressure to the hyperemic pressure, estimate the hyperemic average blood flow and CFR. Finally, based on the hyperemic coronary artery ostium pressure Pa, flow resistance, and hyperemic average blood flow, estimate FFR and IMR. In the following sections, each embodiment of this application will provide detailed descriptions of steps S100 to S700.

In one embodiment, step S100 includes obtaining the resting coronary artery ostium pressure and calculating the cardiac output pressure based on the resting coronary pressure.

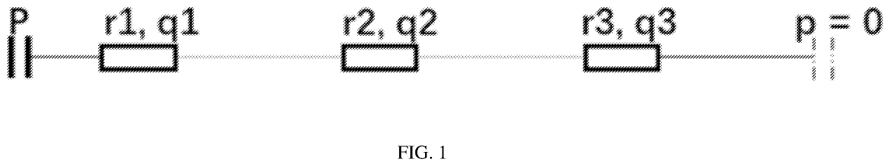

In the coronary circulation, blood flows out of the left ventricle, enters the aorta, and then enters the coronary artery system, finally achieving nutrient and oxygen exchange with the venous system at the microvascular end of the coronary circulation. Therefore, the entire coronary circulation system consists of four parts, namely the cardiac pump source, the aortic segment, the epicardial vessel segment, and the endocardial microvascular segment, with the specific hemodynamic model shown in FIG. 1.

As shown in the FIG., blood flows out of the cardiac pump (with a pump pressure denoted as P), first enters the aorta (with a flow rate denoted as q1 and a flow resistance denoted as r1), then enters the epicardial vessel segment of the coronary artery system (with an average blood flow rate denoted as q2 and a flow resistance denoted as r2), and finally achieves nutrient and oxygen exchange with the venous system through the endocardial microvascular segment (with an average blood flow rate denoted as q3 and a flow resistance denoted as r3).

It is worth noting that due to the existence of various branches in the coronary artery system, q1, q2, and q3 are not equal, but in the entire coronary circulation system, the pressure eventually drops from the cardiac output pressure P to the central venous pressure of approximately 0 mmHg.

After obtaining the resting coronary artery ostium pressure, the first step of this method is to calculate the cardiac output pressure. The method for obtaining the cardiac output pressure is as follows:

P=α*Pa_rest, where: Equation 1

P is the cardiac output pressure.

α is the pressure correction coefficient.

Pa_rest is the resting pressure of the coronary artery ostium.

It can be understood that the resting pressure Pa_rest of coronary artery ostium is the clinical measurement result. In this embodiment, the pressure correction coefficient is related to the normal resting pressure and normal CFR in clinical statistics. Specifically, the pressure correction coefficient α in this method has a range of 1.05 to 1.1.

In one embodiment, step S200 includes obtaining the hyperemic pressure of coronary artery ostium, and calculating the Coronary flow reserve based on the cardiac output pressure, the resting pressure, and the hyperemic pressure of coronary artery ostium. This step is used to calculate the Coronary flow reserve (CFR) and the maximum hyperemic blood flow in the epicardial vessels.

When the coronary vessels transition from the resting state to the hyperemic state, the blood flow and resistance of the three segments of the coronary system are denoted as Q1 and R1, Q2 and R2, Q3 and R3, respectively, as shown in FIG. 1. During the transition, the microvascular resistance of the coronary system decreases significantly, from r3 to R3, due to the dilation of the microvessels. Meanwhile, the blood flow q1, q2, and q3 in the coronary system increase from their resting values to Q1, Q2, and Q3, respectively. Clinical studies have shown that the resistance of the epicardial vessels remains nearly constant during this process, i.e., R1=r1.

When the coronary system is in a resting state, the pressure drop in the aortic segment is the resting pressure drop DP1_rest:

DP1_rest=P−Pa_rest, where P is the cardiac output pressure and Pa_rest is the resting pressure of coronary artery ostium.

When the coronary system transitions to the hyperemic state, the pressure drop in the aortic segment is the hyperemic pressure drop DP1_hyp:

DP1_hyp=P−Pa_hyp, where P is the cardiac output pressure and Pa_hyp is the hyperemic pressure of coronary artery ostium. It can be understood that Pa_hyp is the clinical measurement result of the hyperemic pressure of coronary artery ostium.

By incorporating the hemodynamic model, we can obtain the following:

$$\frac{Q 1}{q 1} = \frac{DP {1}_{hyp}}{DP {1}_{rest}} = \frac{P - Pa\_hyp}{P - Pa\_rest} , \qquad Equation 2$$

where,

Q1 is the blood flow in the aorta in hyperemia.

q1 is the blood flow in the aorta in resting.

P is the cardiac pressure.

Pa_hyp is the hyperemic pressure of the coronary artery ostium.

Pa_rest is the resting pressure of the coronary artery ostium.

Therefore, the calculation formula for CFR of the entire coronary artery system is:

CFR=(P−Pa_hyp)/(P−Pa_rest)

In one embodiment, step S300 includes obtaining medical images of the coronary artery system, extracting the vessel boundary from the medical images, obtaining the three-dimensional model of the target vessel, and calculating the morphological parameters based on the three-dimensional model of the target vessel.

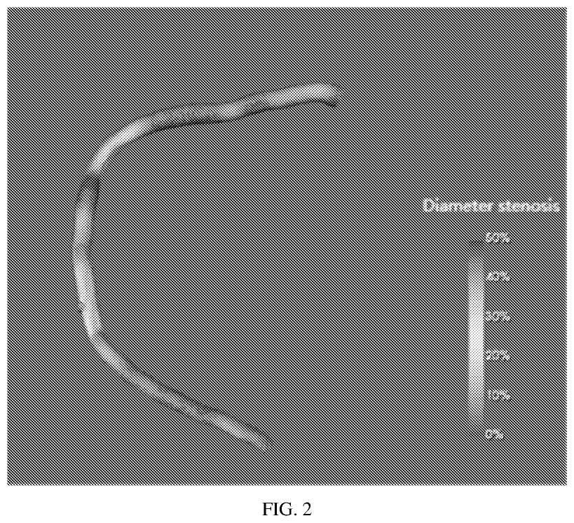

This step is used to process medical images of the coronary artery system, which can be CTA, DSA, MRA, etc. Based on the above medical images, threshold segmentation, feature extraction, dynamic contour, Level-Set, and other methods can be applied to extract the vessel boundary. Finally, the MarchingCube method or binocular vision method is applied to obtain the three-dimensional model of the coronary artery system or target vessel, as shown in FIG. 2. The morphological parameters of the coronary artery can then be calculated, such as the length, diameter, and area of the vessel at each cross-section.

In one embodiment, step S400 includes calculating the resting blood flow based on the morphological parameters. This step is used to calculate the average resting blood flow q2 of the epicardial vessels of the coronary artery system. The methods that can be used include the TIMI frame counting method, rasterization method, myocardial mass estimation method, etc.

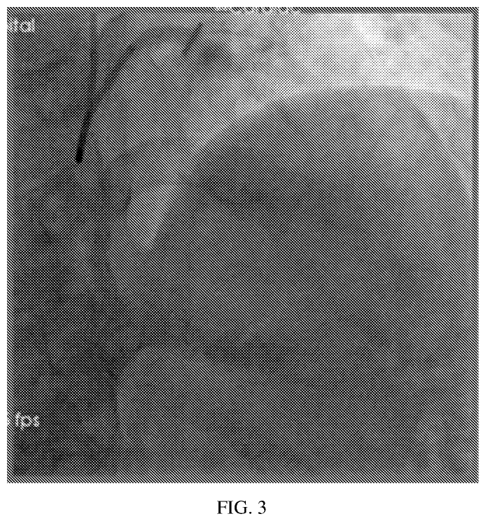

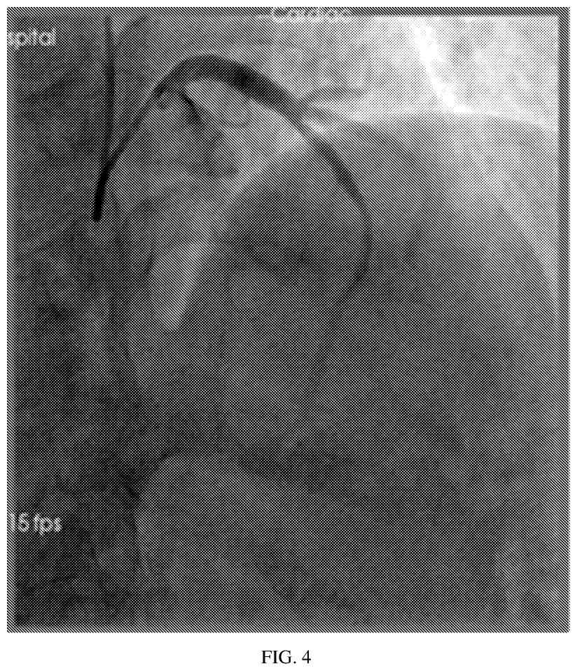

For example, when the input image is a DSA image, this step identifies the starting and ending frames of the image through the TIMI frame counting method, combines the target vessel length L, the average vessel cross-sectional area S, and the parameters of the images, the average blood flow q2 (i.e., resting blood flow) is obtained. The starting frame corresponds to the image frame number when the contrast agent flows out of the catheter and just reaches the starting point of the target vessel, as shown in FIG. 3. The end frame corresponds to the image frame number when the contrast agent just reaches the ending point of the target vessel, as shown in FIG. 4. The calculation method of the average resting blood flow is as follows:

q2=S*L/((F2−F1)/fps), where: Equation 3

q2 is the resting blood flow.

S is the average vessel cross-sectional area.

L is the length of the target vessel.

F2 is the ending frame.

F1 is the starting frame.

fps is the time resolution of the images.

In one embodiment, step S500 includes calculating the flow resistance based on the morphological parameters and resting blood flow. This step includes calculating the viscosity resistance and expansion resistance based on the morphological parameters, and then combining the resting blood flow to obtain the flow resistance.

This step is used to calculate the flow resistance r2 of the epicardial vessels of the coronary artery system. After the three-dimensional model of the coronary vessel is reconstructed to obtain the morphological parameters, the flow resistance r2 can be calculated by combining the average blood flow q2 (as described in step S400 for resting blood flow) in the epicardial vessels of the coronary artery system with the following method:

r2=rᵥ+rₑ*q2, where: Equation 4

r2 is the flow resistance.

rᵥ is the viscosity resistance, which is related to the vessel morphology.

rₑ is the expansion resistance, which is related to the degree of vessel stenosis.

q2 is the resting blood flow.

Specifically, the calculation formulas for the two resistance components are:

$${r}_{v} = \frac{8 μ L}{π {R}^{4}} ,$$

where μ is the blood viscosity and R is the radius of the vessel.

$${r}_{e} = \frac{p {K}_{e}}{2 {A}_{0}^{2}} {( \frac{{A}_{0}}{{A}_{s}} - 1 )}^{2} ,$$

where ρis blood density, A₀ and Aₛ are the reference vessel area and stenosis area, respectively, and Kₑ is the expansion resistance correction factor related to the stenosis length, which ranges from 1.2 to 2.1.

In one embodiment, step S600 includes: calculating the average blood flow of the epicardial vessels in hyperemia based on the Coronary flow reserve and resting blood flow, and calculating the Fractional Flow Reserve and the distal pressure of the target vessel in hyperemia based on morphological parameters, the average blood flow in hyperemia, and the hyperemic pressure of the coronary artery ostium.

Specifically, the viscous and expansion resistance of the target vessel are calculated based on the morphological parameters, and the distal pressure of the target vessel in hyperemia and the Fractional flow reserve are calculated based on the hyperemic pressure of the coronary artery ostium.

This step is used to calculate the Fractional flow reserve (FFR) of the target vessel and the distal pressure Pd of the target vessel in hyperemia. According to the CFR definition, the average blood flow of the epicardial vessels in hyperemia can be defined as:

Q2=CFR*q2, where: Equation 5

Q2 is the average blood flow in hyperemia.

q2 is the resting blood flow.

In the calculation of CFR, we obtain the average blood flow Q2 of the epicardial vessels in hyperemia. Since there is no significant morphological change in the epicardial vessels, i.e., rᵥ and rₑ remain approximately constant, the resistance of the epicardial vessels in hyperemia can be approximated as: R2=rᵥ+rₑ*Q2.

Based on the hemodynamic model, the pressure drop of the epicardial vessels can be approximated as:

DP2=(rᵥ+rₑ*Q2)*Q2, where:

DP2 is the pressure drop of the epicardial vessels.

rᵥ is the viscous resistance related to the vessel morphology.

rₑ is the expansion resistance related to the degree of vessel stenosis.

Q2 is the average blood flow in hyperemia.

According to the FFR definition, the FFR of the target vessel can be calculated as:

FFR=Pd_hyp/Pa_hyp=(Pa_hyp−(rᵥ+rₑ*Q2)*Q2)/Pa_hyp, Equation 6

where Pd_hyp is the distal pressure of the target vessel in hyperemia, and Pa_hyp is the hyperemic pressure of the coronary artery ostium.

In one embodiment, step S700 includes: calculating the Index of Microvascular Resistance (IMR) based on morphological parameters, the average blood flow Q2 of the epicardial vessel in hyperemia, and the pressure at the distal end of the target vessel.

This step is used to calculate both the IMR and the mean transit time (Tmn). During the reconstruction of the 3D model, the length and average cross-sectional area of the target vessel are obtained. During the calculation of Coronary flow reserve (CFR), the average blood flow Q2 of the target vessel in hyperemia is obtained. During the calculation of Fractional flow reserve (FFR), the distal pressure of the target vessel in hyperemia (Pd_hyp) is obtained.

Firstly, we need to calculate the mean transit time Tmn:

Tmn=L/(Q2/S)

where L is the length of the target vessel, Q2 is the average blood flow of the target vessel in hyperemia, and S is the average cross-sectional area of the target vessel.

Then, according to the definition of IMR, the Index of Microvascular Resistance of the target vessel can be approximated as:

IMR=S*L*Pd_hyp/Q2

where S is the average cross-sectional area of the target vessel, L is the length of the target vessel, Pd_hyp is the distal pressure of the target vessel in hyperemia, and Q2 is the average blood flow of the target vessel in hyperemia.

To verify the reliability of the algorithms provided in the embodiments of the present application, two sets of experiments are conducted as follows.

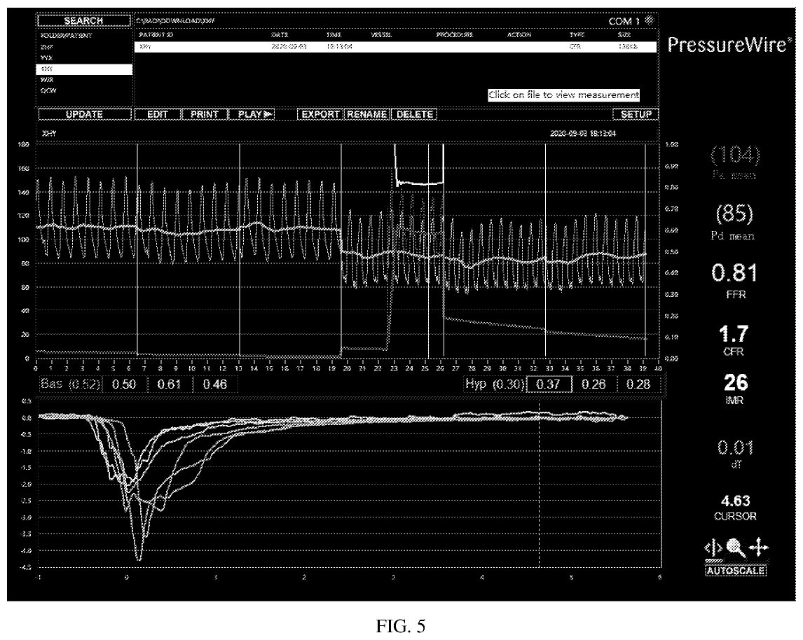

In one embodiment, IMR is calculated and verified. The input of this embodiment is data achieved in hyperemic state, including angiographic images in the state of hyperemia and coronary artery ostium pressure Pa. The measured values in the catheter chamber are shown in FIG. 5, where the coronary artery ostium pressure Pa in the hyperemia state is 104 mmHg, the distal pressure of the stenosis Pd is 85 mmHg, and the average transit time Tmn is 0.3 s. The measured value of IMR is:

IMR=85*0.3=25.5.

For this case, the method provided by the present application is used to calculate the Index of Microvascular Resistance. In the calculation process of this case, DP=16.8 mmHg and Tmn=0.2933 s. The calculated IMR value is:

IMR=(104−16.8)*0.2933=25.58.

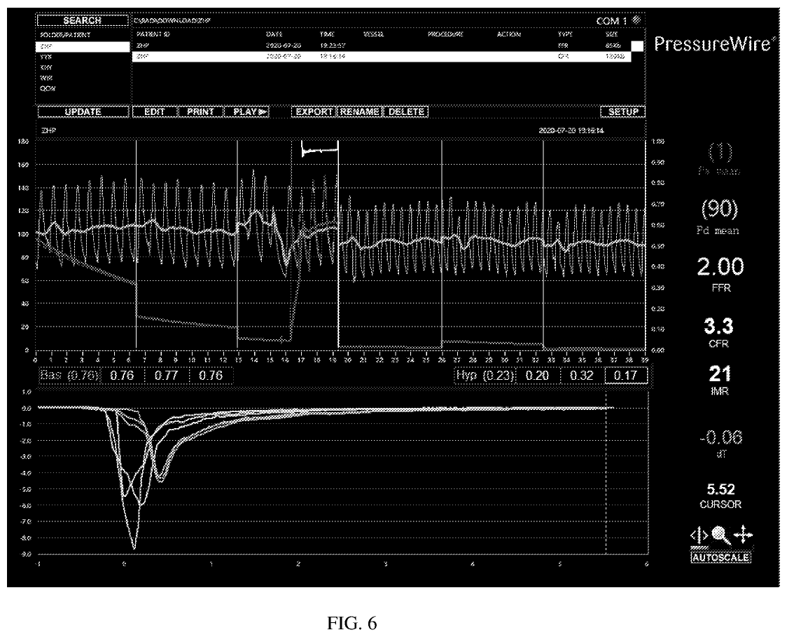

In another embodiment, IMR is calculated and verified. The input of this embodiment is data achieved in resting state, including angiographic images in the resting state and coronary artery ostium pressure. The measured values in the catheter chamber are shown in FIG. 6, where the distal pressure of the stenosis Pd in the hyperemic state is 90 mmHg, and the average transit time Tmn is 0.23 s. The measured value of IMR is:

IMR=90*0.23=20.7.

For this case, the method provided the present application is used to calculate the Index of Microvascular Resistance. In the calculation process of this case, the coronary artery ostium pressure in the resting state is 113 mmHg, the coronary artery ostium pressure in the hyperemic state Pa is 96 mmHg, the pressure drop DP is 5.7 mmHg, Tmn is 0.2103 s, and the calculated IMR value is:

IMR=(96−5.7)*0.2103=18.99.

Based on the above experiments, it can be concluded that since the value of the Index of Microvascular Resistance is the result of step S700, it also proves the accuracy of the CFR and FFR obtained in the intermediate steps.

It should be understood that although the steps in the flowchart of FIG. 7 are shown sequentially according to the direction of the arrows, these steps are not necessarily executed in the order indicated by the arrows. Unless otherwise specified in this document, the execution of these steps is not strictly limited to a particular order and can be performed in a different sequence. Moreover, at least some of the steps in FIG. 7 may include multiple sub-steps or stages, which are not necessarily completed at the same time and may be executed at different times. The execution order of these sub-steps or stages is not necessarily sequential, but can be performed alternately or intermittently with at least some of the sub-steps or stages of other steps or other steps.

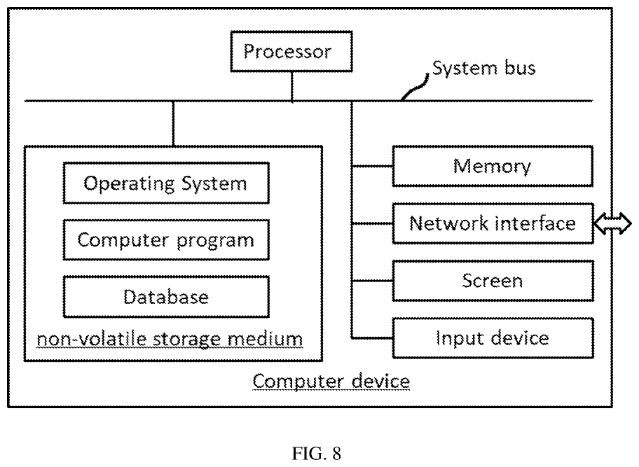

In one embodiment, a computer device is provided for obtaining the Index of Microvascular Resistance. The computer device can be a terminal, as shown in FIG. 8, and includes a processor, memory, network interface, display, and input device connected through a system bus. The processor of the computer device provides computing and control capabilities. The memory of the computer device includes non-volatile storage media and internal memory. The non-volatile storage media stores an operating system and computer programs. The internal memory provides an environment for the operation of the operating system and computer programs stored in the non-volatile storage media. The network interface of the computer device is used to communicate with external terminals through a network. When executed by the processor, the computer program implements a method for obtaining the Index of Microvascular Resistance. The display of the computer device can be a liquid crystal display or an electronic ink display, and the input device of the computer device can be a touch layer on the display screen, buttons, trackballs or touchpads on the computer device housing, or external keyboards, touchpads, or mice.

In one embodiment, a computer device is provided, comprising a memory and a processor, wherein the memory stores computer programs and the processor executes the computer programs to implement the following steps:

Step S100, obtaining the resting pressure of the coronary artery ostium, and calculating the cardiac output pressure based on the resting pressure of the coronary artery ostium.

Step S200, obtaining the hyperemic pressure of the coronary artery ostium, and obtaining the Coronary flow reserve based on the cardiac output pressure, the resting pressure of the coronary ostium, and the hyperemic pressure of the coronary artery ostium.

Step S300, obtaining medical images of the coronary artery system, extracting the vascular boundary of the coronary artery from the medical images, obtaining the target vessel's 3D model, and calculating the morphological parameters of the coronary artery based on the 3D model of the target vessel.

Step S400, calculating the resting blood flow based on the morphological parameters.

Step S500, calculating the flow resistance based on the morphological parameters and the resting blood flow.

Step S600, calculating the average blood flow of the epicardial vessels in hyperemia based on the Coronary flow reserve and resting blood flow, and calculating the Fractional Flow Reserve and the pressure at the distal of the target vessel in hyperemia based on the morphological parameters, average blood flow in hyperemia, and hyperemic pressure of the coronary artery ostium.

Step S700, calculating the Index of Microvascular Resistance based on the morphological parameters, average blood flow of the epicardial vessels in hyperemia, and pressure at the distal of the target vessel.

In one embodiment, when the processor executes the computer programs, the following steps are also implemented:

Step S100, obtaining the resting pressure of the coronary artery ostium, and calculating the cardiac output pressure based on the resting pressure of the coronary artery ostium.

Step S200, obtaining the hyperemic pressure of the coronary artery ostium, and obtaining the Coronary flow reserve based on the cardiac output pressure, the resting pressure of the coronary ostium, and the hyperemic pressure of the coronary artery ostium.

Step S300, obtaining medical images of the coronary artery system, extracting the vascular boundary of the coronary artery from the medical images, obtaining the target vessel's 3D model, and calculating the morphological parameters of the coronary artery based on the 3D model of the target vessel.

Step S400, calculating the resting blood flow based on the morphological parameters.

Step S500, calculating the flow resistance based on the morphological parameters and the resting blood flow.

Step S600, calculating the average blood flow of the epicardial vessels in hyperemia based on the Coronary flow reserve and resting blood flow, and calculating the Fractional Flow Reserve and the pressure at the distal of the target vessel in hyperemia based on the morphological parameters, average blood flow in hyperemia, and hyperemic pressure of the coronary artery ostium.

Step S700, calculating the Index of Microvascular Resistance based on the morphological parameters, average blood flow of the epicardial vessels in hyperemia, and pressure at the distal of the target vessel.

The present embodiment provides a computer storage medium on which computer programs are stored. When the computer programs are executed by a processor, the following steps are implemented:

Step S100, obtaining the resting pressure of the coronary artery ostium, and calculating the cardiac output pressure based on the resting pressure of the coronary artery ostium.

Step S200, obtaining the hyperemic pressure of the coronary artery ostium, and obtaining the Coronary flow reserve based on the cardiac output pressure, the resting pressure of the coronary ostium, and the hyperemic pressure of the coronary artery ostium.

Step S300, obtaining medical images of the coronary artery system, extracting the vascular boundary of the coronary artery from the medical images, obtaining the target vessel's 3D model, and calculating the morphological parameters of the coronary artery based on the 3D model of the target vessel.

Step S400, calculating the resting blood flow based on the morphological parameters.

Step S500, calculating the flow resistance based on the morphological parameters and the resting blood flow.

Step S600, calculating the average blood flow of the epicardial vessels in hyperemia based on the Coronary flow reserve and resting blood flow, and calculating the Fractional Flow Reserve and the pressure at the distal of the target vessel in hyperemia based on the morphological parameters, average blood flow in hyperemia, and hyperemic pressure of the coronary artery ostium.

Step S700, calculating the Index of Microvascular Resistance based on the morphological parameters, average blood flow of the epicardial vessels in hyperemia, and pressure at the distal of the target vessel.

In one embodiment, a computer program product is provided, comprising computer instructions that, when executed by a processor, perform the following steps:

Step S100, obtaining the resting pressure of the coronary artery ostium, and calculating the cardiac output pressure based on the resting pressure of the coronary artery ostium.

Step S200, obtaining the hyperemic pressure of the coronary artery ostium, and obtaining the Coronary flow reserve based on the cardiac output pressure, the resting pressure of the coronary ostium, and the hyperemic pressure of the coronary artery ostium.

Step S300, obtaining medical images of the coronary artery system, extracting the vascular boundary of the coronary artery from the medical images, obtaining the target vessel's 3D model, and calculating the morphological parameters of the coronary artery based on the 3D model of the target vessel.

Step S400, calculating the resting blood flow based on the morphological parameters.

Step S500, calculating the flow resistance based on the morphological parameters and the resting blood flow.

Step S600, calculating the average blood flow of the epicardial vessels in hyperemia based on the Coronary flow reserve and resting blood flow, and calculating the Fractional Flow Reserve and the pressure at the distal of the target vessel in hyperemia based on the morphological parameters, average blood flow in hyperemia, and hyperemic pressure of the coronary artery ostium.

Step S700, calculating the Index of Microvascular Resistance based on the morphological parameters, average blood flow of the epicardial vessels in hyperemia, and pressure at the distal of the target vessel.

In this embodiment, the computer program product includes a program code portion for executing the steps for obtaining Coronary flow reserve, obtaining Fractional flow reserve, or obtaining the Index of Microvascular Resistance as described in various embodiments of this application, when the computer program product is executed by one or more computing devices. The computer program product can be stored on computer-readable recording media. The computer program product can also be provided for download via a data network (e.g., via RAN, over the Internet, and/or via RBS). Alternatively or additionally, the method can be encoded in field-programmable gate arrays (FPGAs) and/or application-specific integrated circuits (ASICs), or provided functionally by a hardware description language for download.

The ordinary skilled person in this field can understand that all or part of the processes for implementing the above-described embodiments can be accomplished by instructing related hardware through a computer program. The computer program can be stored in a non-volatile computer-readable storage medium, and when executed, the computer program can include the processes of the embodiments described above. Any reference to memory, storage, database, or other media used in the various embodiments provided in this application can include non-volatile and/or volatile memory. Non-volatile memory can include read-only memory (ROM), programmable ROM (PROM), electrical programmable ROM (EPROM), electrical erasable programmable ROM (EEPROM), or flash memory. Volatile memory can include random access memory (RAM) or external high-speed cache memory. As an illustration rather than a limitation, RAM can take various forms such as static RAM (SRAM), dynamic RAM (DRAM), synchronous DRAM (SDRAM), double data rate SDRAM (DDRSDRAM), enhanced SDRAM (ESDRAM), synchronous link (Synchlink) DRAM (SLDRAM), Rambus direct RAM (RDRAM), direct RDRAM (DRDRAM), and Rambus dynamic RAM (RDRAM), etc.

The various technical features of the above embodiments can be arbitrarily combined. In order to keep the description concise, not all possible combinations of the technical features in the above embodiments are described. However, as long as the combination of these technical features is not contradictory, it should be considered within the scope of this specification. When the technical features of different embodiments are reflected in the same FIG., it can be regarded as the disclosure of the combination examples of all the involved embodiments in that FIG.

The above embodiments only express several embodiments of the present application, which are described in a specific and detailed manner. However, this should not be understood as a limitation on the scope of the invention. It should be pointed out that for those skilled in the field, various modifications and improvements can be made without departing from the concept of the present application, and these all fall within the scope of protection of the present application. Therefore, the scope of protection of the present patent application should be determined by the claims attached hereto.

1. The method for obtaining the Index of Microvascular Resistance comprises:
obtaining the resting pressure of coronary artery ostium, calculating cardiac output pressure according to the obtained resting pressure of coronary artery ostium;
obtaining the hyperemic pressure of the coronary artery ostium, obtaining the coronary flow reserve based on the cardiac output pressure, the resting pressure of coronary artery ostium, and the hyperemic pressure of the coronary artery ostium;
obtaining the medical images of the coronary system, extracting the vessel boundary from the images to obtain a target vessel 3D model, and calculating the morphological parameters of the coronary artery according to the 3D model of the target vessel;
calculating the resting blood flow based on the morphological parameters;
calculating the flow resistance based on the morphological parameters and the resting blood flow;
calculating the average blood flow of the epicardial vessels in hyperemia based on the Coronary flow reserve and the resting blood flow, calculating the Fractional flow reserve and the pressure at the distal of the target vessel in hyperemia based on the morphological parameters, the average blood flow in hyperemia, and the hyperemic pressure of the coronary artery ostium; and
calculating the Index of Microvascular Resistance based on the morphological parameters, the average blood flow in hyperemia, and the pressure at the distal of the target vessel.

2. The method for obtaining the Index of Microvascular Resistance according to claim 1, wherein the cardiac output pressure is obtained by the following formula:
P=a*Pa_rest, where:
P is the cardiac output pressure;
a is the pressure correction factor; and
Pa_rest is the resting pressure of the coronary artery ostium.

3. The method for obtaining the Index of Microvascular Resistance according to claim 2, wherein the Coronary flow reserve is obtained by the following formula:
$$CFR = \frac{P - Pa\_hyp}{P - Pa\_rest} ,$$
where:
CFR is the Coronary flow reserve; and
Pa_hyp is the hyperemic pressure of the coronary artery ostium.

4. The method for obtaining the Index of Microvascular Resistance according to claim 1, wherein based on mentioned morphological parameters and mentioned resting blood flow, the flow resistance is calculated, specifically comprising:
obtaining the viscous resistance and expansion resistance based on mentioned morphological parameters, and calculating the flow resistance by combining with mentioned resting blood flow, mentioned morphological parameters include: target vessel length, vessel radius, reference vessel area, mean vessel cross-sectional area, and narrowed vessel area, the flow resistance is obtained by the following formula:
r2=rᵥ+rₑ*q2, where:
r2 is the flow resistance;
rᵥ is the viscous resistance, which is related to the vessel morphology.
rₑ is the expansion resistance, which is related to the degree of vessel stenosis;
q2 is the resting blood flow;
the viscous resistance is obtained by the following formula:
$${r}_{v} = \frac{8 μ L}{π {R}^{4}} ,$$
where:
μ is the blood viscosity coefficient;
R is the vessel radius;
L is the target vessel length;
the expansion resistance is obtained by the following formula:
$${r}_{e} = \frac{p {K}_{e}}{2 {A}_{0}^{2}} {( \frac{{A}_{0}}{{A}_{s}} - 1 )}^{2} ,$$
where:
ρis the blood density;
A₀ and Aₛ are the reference and stenotic cross-sectional areas, respectively; and
Kₑ is a expansion resistance correction coefficient related to the stenosis length, with a range of 1.2 to 2.1.

5. The method for obtaining the Index of Microvascular Resistance according to claim 4, wherein in that, specifically comprising:
obtaining the pressure drop of the epicardial vessels based on the average blood flow of the epicardial vessels in hyperemia, viscous resistance, and expansion resistance, the pressure drop of the epicardial vessels is obtained by the following formula:
DP2=(rᵥ+rₑ*Q2)*Q2, where:
DP2 is the pressure drop of the epicardial vessels;
rᵥ is the viscous resistance, which is related to the vessel morphology;
rₑ is the expansion resistance, which is related to the degree of vessel stenosis; and
Q2 is the average blood flow in hyperemia.

6. The method for obtaining the Index of Microvascular Resistance according to claim 4, wherein the Fractional Flow Reserve is obtained by the following formula:
FFR=(Pa_hyp−(rᵥ+rₑ*Q2)*Q2)/Pa_hyp, where:
rᵥ is the viscous resistance, which is related to the vessel morphology;
rₑ is the expansion resistance, which is related to the degree of vessel stenosis;
Q2 is the average blood flow in hyperemia; and
Pa_hyp is the hyperemic pressure of the coronary artery ostium.

7. The method for obtaining the Index of Microvascular Resistance according to claim 4, wherein the Index of Microvascular Resistance is obtained by the following formula:
IMR=S*L*Pd_hyp/Q2, where:
IMR is the Index of Microvascular Resistance;
S is the average cross-sectional area of the target vessel;
L is the length of the target vessel;
Pd_hyp is the pressure at the distal of the target vessel in hyperemia; and
Q2 is the average blood flow in hyperemia.

8. The computer equipment for obtaining the Index of Microvascular Resistance, comprises a memory, a processor, and computer programs stored in the memory, wherein the processor executes the computer programs to implement the steps of claim 1 for obtaining the Index of Microvascular Resistance.

9. A computer storage medium, on which computer programs are stored, characterized in that, when the computer programs are executed by a processor, the steps of claim 1 for obtaining the Index of Microvascular Resistance are implemented.

10. A computer program product, comprises computer instructions, characterized in that, when the computer instructions are executed by a processor, the steps of claim 1 for obtaining the Index of Microvascular Resistance are implemented.

11. The computer equipment for obtaining the Index of Microvascular Resistance, comprises a memory, a processor, and computer programs stored in the memory, wherein the processor executes the computer programs to implement the steps of claim 2 for obtaining the Index of Microvascular Resistance.

12. The computer equipment for obtaining the Index of Microvascular Resistance, comprises a memory, a processor, and computer programs stored in the memory, wherein the processor executes the computer programs to implement the steps of claim 3 for obtaining the Index of Microvascular Resistance.

13. The computer equipment for obtaining the Index of Microvascular Resistance, comprises a memory, a processor, and computer programs stored in the memory, wherein the processor executes the computer programs to implement the steps of claim 4 for obtaining the Index of Microvascular Resistance.

14. The computer equipment for obtaining the Index of Microvascular Resistance, comprises a memory, a processor, and computer programs stored in the memory, wherein the processor executes the computer programs to implement the steps of claim 5 for obtaining the Index of Microvascular Resistance.

15. The computer equipment for obtaining the Index of Microvascular Resistance, comprises a memory, a processor, and computer programs stored in the memory, wherein the processor executes the computer programs to implement the steps of claim 6 for obtaining the Index of Microvascular Resistance.

16. The computer equipment for obtaining the Index of Microvascular Resistance, comprises a memory, a processor, and computer programs stored in the memory, wherein the processor executes the computer programs to implement the steps of claim 7 for obtaining the Index of Microvascular Resistance.

17. The computer storage medium of claim 9, wherein the cardiac output pressure is obtained by the following formula:
P=a*Pa_rest, where:
P is the cardiac output pressure;
a is the pressure correction factor; and
Pa_rest is the resting pressure of the coronary artery ostium.

18. The computer storage medium of claim 17, wherein the Coronary flow reserve is obtained by the following formula:
$$CFR = \frac{P - Pa\_hyp}{P - Pa\_rest} ,$$
where:
CFR is the Coronary flow reserve; and
Pa_hyp is the hyperemic pressure of the coronary artery ostium.

19. The computer storage medium of claim 9, based on mentioned morphological parameters and mentioned resting blood flow, the flow resistance is calculated, specifically comprising:
obtaining the viscous resistance and expansion resistance based on mentioned morphological parameters, and calculating the flow resistance by combining with mentioned resting blood flow, mentioned morphological parameters include: target vessel length, vessel radius, reference vessel area, mean vessel cross-sectional area, and narrowed vessel area, the flow resistance is obtained by the following formula:
r2=rᵥ+rₑ*q2, where:
r2 is the flow resistance;
rᵥ is the viscous resistance, which is related to the vessel morphology.
rₑ is the expansion resistance, which is related to the degree of vessel stenosis;
q2 is the resting blood flow;
the viscous resistance is obtained by the following formula:
$${r}_{v} = \frac{8 μ L}{π {R}^{4}} ,$$
where:
μ is the blood viscosity coefficient;
R is the vessel radius;
L is the target vessel length;
the expansion resistance is obtained by the following formula:
$${r}_{e} = \frac{p {K}_{e}}{2 {A}_{0}^{2}} {( \frac{{A}_{0}}{{A}_{s}} - 1 )}^{2} ,$$
where:
ρis the blood density;
A₀ and Aₛ are the reference and stenotic cross-sectional areas, respectively; and
Kₑ is a expansion resistance correction coefficient related to the stenosis length, with a range of 1.2 to 2.1.

20. The computer storage medium of claim 19, wherein specifically comprising:
obtaining the pressure drop of the epicardial vessels based on the average blood flow of the epicardial vessels in hyperemia, viscous resistance, and expansion resistance, the pressure drop of the epicardial vessels is obtained by the following formula:
DP2=(rᵥ+rₑ*Q2)*Q2, where:
DP2 is the pressure drop of the epicardial vessels;
rᵥ is the viscous resistance, which is related to the vessel morphology;
rₑ is the expansion resistance, which is related to the degree of vessel stenosis; and
Q2 is the average blood flow in hyperemia.
